(19) United States
(12) Patent Application Publication
(10) Pub. No.: US 2009/0247646 A1
(43) Pub. Date: Oct. 1, 2009

(54) USE OF RESVERATROL AND DERIVATIVES THEREOF FOR PROMOTING THE WELLNESS STATE IN MAMMALS

(76) Inventors: Daniel Raederstorff, Flaxlanden (FR); Volker Schehlmann, Schopfheim (DE); Ying Wang-Schmidt, Stallikon (CH); Gilbert Weber, Magden (CH)

Correspondence Address: NIXON & VANDERHYE, PC, 901 NORTH GLEBE ROAD, 11TH FLOOR, ARLINGTON, VA, 22203, US

(21) Appl. No.: 12/280,417

(22) PCT Filed: Feb. 14, 2007
(86) PCT No.: PCT/EP2007/001256

§ 371 Date: Mar. 20, 2009

(30) Foreign Application Priority Data
Feb. 24, 2006 (EP) 06003757.9
May 15, 2006 (EP) 06009942.1

(51) Int. Cl. A61K 31/047; C07C 39/21

(52) U.S. Cl. 514/729; 568/729

(57) ABSTRACT

The invention relates to the use of resveratrol, a derivative, metabolite or analogue thereof for promoting the wellness state of a mammal or for changing gene expression profiles in older adult mammals towards conformity with expression profiles found in younger adult mammals as well as to their use for the manufacture of corresponding nutraceutical compositions.

The present invention refers to a method for promoting the wellness state of an organism by inducing physiological changes that will give the organism a younger molecular phenotype. The expression “organism” is to be understood to refer to mammals which comprises human beings and animals. Mammals in the context of the present invention include humans, other primates, carnivores, artiodactyla, rodents, perissodactyla, lagomorpha etc. Preferred “mammals” are humans and pets such as cats, dogs and horses.

Wellness denotes that an organism is able to function optimally under the conditions of its environment. It includes optimal physical and mental functions. Thus, wellness is defined as a state of general good body function in which all functions of an organism are in optimal working conditions, being free of activity limitations and chronic disorders. Thus, mobility, physical performance and tolerance to thermal stress are maintained. The organism is free from physical disorders, is not damaged and free from pain. It is able to cope with everyday activities and has the flexibility to deal with life's inevitable challenges, thus being physically fit for a high quality of life. In addition, it is in a state of optimal performance of mental function, resulting in learning and productive activities, it has social connectivity to other individuals and the ability to adapt to changes and to cope with adversity and stress. It is also free from physical alteration and mental problems such as depression, anxiety, cognitive problems, and alterations in thinking, mood or behavior.

Differences in the wellness of an organism can most strikingly be observed if one compares a young organism with an old organism. A difference in the wellness state between a young and an old organism is reflected in differences in their gene expression profiles which can be used as a molecular signature of wellness. The state of wellness can be determined, inter alia, by evaluating measurable parameters of body functions, such as, e.g., blood pressure, heart rate, body fat composition, pulmonary function, liver function, brain function and levels of physiologically vital components in body fluids, etc., as disclosed in U.S. Pat. No. 5,692,501.

Promotion of the wellness state, in the context of the present invention, denotes an improvement in the general health state of an organism including both physical and mental health and is achieved by inducing physiological changes represented by a change in the gene expression profile of an adult organism typical of higher age to that of the same organism but of lower age. The effect of promotion of the wellness state of an organism in accordance with the present invention is a rejuvenating effect resulting in a younger phenotype. This rejuvenation effect is different from an anti-aging effect and corresponding treatments which are aimed at delaying the aging process in an organism and ultimately trying to extend its lifespan by preventing loss of physiological functions and age-related diseases. In contrast, the wellness-promoting treatment aims to restore the wellness state of an older organism, thus making it identical or more similar to the wellness state of an equivalent, younger organism.

The terms “old” or “older” and “young” or “younger” do not represent absolute values but are relative, individual terms; they are typical for each organism species.

Key factors for the wellness state of an organism are, e.g., the capacity of an organism to maintain homeostasis and its capacity for repair and regeneration.

Homeostasis is the inherent tendency of an organism towards maintenance of physiological and psychological stability. Homeostatic mechanisms in mammals are for example: the regulation of body temperature, the regulation of the amounts of water and minerals in the body, the removal of metabolic waste, the regulation of blood glucose and lipid levels. At the cellular level, factors such as temperature, salinity, acidity, concentrations of nutrients, such as glucose, lipids, various ions (calcium, sodium, potassium, etc.), oxygen and wastes, such as carbon dioxide and urea must also be maintained within tolerable limits. In multi-cellular organisms those factors also have to be maintained at desirable levels in body fluids such as blood plasma, tissue fluid and intracellular fluid to allow the organism to function more effectively. Complex systems, such as the human body, must therefore have efficient homeostatic mechanisms to be able to adapt to modifications of the environment and to maintain stability and a state of wellness in the body. Disruptions in above-mentioned factors will have a negative impact on metabolism, which constitutes all the biochemical processes of an organism keeping it healthy and alive. Such a dysregulation of cellular homeostasis will then induce a gradual decline of organ functions. Therefore, there are in-built physiological mechanisms (homeostatic systems) to maintain the factors at desirable levels. Homeostasis is not a static state but a state of equilibrium. At the cellular level homeostasis must be maintained in the presence of a constant metabolic flux of molecules. For example, cellular components such as proteins, lipid membranes, sugars and nucleic acids, are constantly recycled while the integrity of the organism as a whole is preserved by homeostatic systems. In an organism challenged by multiple internal and external stimuli the homeostatic mechanism must be robust and stable to preserve the proper functioning of its component cells, organs, organ systems and whole body. Thus homeostatic mechanisms are essential for maintenance of the internal environment of an organism within tolerable limits to sustain health and optimal function. This occurs by regulating the metabolism of an organism through numerous metabolic pathways which are series of chemical reactions occurring in cells or whole organisms. The metabolism of an organism can be divided in two main processes: (1) the biosynthesis of molecules (anabolism) in which cells use energy to build complex molecules, cellular structure and perform the organism functions and (2) the process for breaking down molecules to produce energy (catabolism). The whole organism must also maintain homeostasis between catabolism and anabolism.

If an organism loses its normal homeostasis, adverse symptoms develop and mechanisms are activated in an attempt to restore balance. If this is not successful, over time disorders will develop. Therefore, chronic imbalances of normal metabolic pathways result in the development of numerous disorders.

The regulation of metabolism and metabolic pathways to maintain homeostasis are central to the molecular events resulting in health and optimal function or, alternatively, disorders. Cellular metabolism is linked to cellular health, and the wellness of an organism is correlated with cellular function in that organism. Cellular metabolism is maintained in a state of dynamic equilibrium (homeostasis) by interrelated complex metabolic pathways. The cellular responses to stimuli are often a result of coordinated activity of groups of genes which tend to maintain homeostasis. Thus, the cells respond to internal and external stimuli by changes in gene expression profiles.

Cellular metabolism can directly and profoundly influence gene expression which, in turn, will affect cellular metabolism by feedback mechanisms. The gene expression profile, therefore, modulates the function of an organism by regulating metabolic pathways within its cells. Thus, a change in gene expression will have an impact on cellular metabolism and vice versa. Gene expression profiles can be used as global markers of the status and response of cells, organs and whole organisms and result from changes in the status of proteins and other metabolites. A gene expression profile can also be used to identify specific metabolic processes and cellular functions which differ in different individuals. Global gene expression profiling will define the genome wide response occurring during cellular metabolism, and any changes in gene expression will reflect changes in cellular function.

Gene expression profiles give a global view of the cellular activities and functions reflecting the physiological and wellness states of an organism.

Microarray technology enables genome-wide gene expression analysis to determine global gene expression in tissues and assess the gene regulation in an organism. Such technology can be used to analyze the expression-pattern and expression-levels of thousands of genes simultaneously. Thus, the transcriptional status of the majority of genes in a particular genome is determined. The technique also enables a direct quantitative comparison of the expression level of specific genes in the same tissues from organisms under different physiological conditions, and will provide information on the physiological state of that organism at the molecular level. Changes in gene expression provide the molecular phenotype of a tissue and can be used to discriminate between normal and pathological states, as well as to determine the effect of certain interventions on such physiological states. When applied to nutritional interventions, microarray technology is a highly effective tool for understanding the function of dietary nutrients and the global response of an organism to those nutrients.

Cellular responses to external stimuli or intracellular fluxes of molecules are often transient but can have a profound impact on cellular function. Modulation of gene expression is an early and rapid response of the cell to challenges and changes in cellular processes. Thus, the coordinated regulation of gene expression is essential for a cell or an organism to respond successfully to external or internal stimuli and to adapt to changing intracellular environments and maintain homeostasis.

A disorder is usually diagnosed by measuring various biomarkers. However, a disorder that results from long-term imbalances in metabolism may not have a measurable biomarker of damage before the disorder is well established. Thus, often when a physician makes a diagnosis the disease is already present. Moreover, it is often not possible to reverse chronic disorders by simply restoring the normal balance of specific aspects of metabolism. A change in gene expression is an early event in reaction to a challenge or change in cell processes, and enables the detection and correction of any undesired structural changes at the molecular level and at a very early stage before any damage has occurred, thus maintaining an organism in an optimal physiological or wellness state.

The key factors known to make an undamaged, healthy young adult organism fitter and maintained in a better state of wellness than an old organism, are a better capacity to maintain homeostasis, more efficient repair systems and a higher regeneration capacity. For example, young organisms have a higher capacity than old organisms to repair and regenerate tissues such as liver, muscle, bone and arterial walls. Young adult organisms are more resistant to stress due to their ability to better maintain cellular homeostasis under challenging conditions, compared with old organisms. Young organisms have sensitive homeostatic mechanisms which, in comparison to older organisms, can respond more rapidly to an imbalance in cellular metabolism and canrepair the damage faster thus also decreasing the recovery period after a challenge. Young adult organisms are also better able to maintain homeostasis due to their wider dynamic range of conditions within which they can function properly, for example, they have better thermo-regulation and are more resistant to a heat shock (thermal stress) than corresponding older organisms. Young organisms also have a higher capacity to maintain homeostasis by more rapidly removing damaged cells and molecules and having a faster repair and regenerative capacity, thus maintaining the body in a better wellness state.

Tissues of the body regenerate well in young individuals, less so in older individuals. Recently, Conboy et al. (Nature 2005, vol. 433: 760-764) investigated whether this decline is irreversible, or whether it can be modulated by factors in the circulation. They joined together the circulatory systems of young and old mice, as a ‘parabiotic’ pair and demonstrated that the regenerative capacity of aged muscle and liver were recovered in the presence of serum from younger animals. They also observed that at the same time there was a restoration of a younger molecular signaling profile. The study showed that tissue regenerative potential can be reversed through the modulation of systemic factors, and suggested that systemic factors can modulate the molecular signaling pathways critical to the activation of the tissue regenerative capacity. Thus, old cells may regain a younger phenotype when exposed to a young cell environment with a younger molecular cellular signaling profile.

The link between the global gene expression profile and the physiological function of an organ has recently been shown in humans by Rodwell et al. (PLOS Biology (2004), 2 (12) 2191-2201). In this study the gene expression profiles correlated well with the morphological and physiological state of the kidney in humans. Moreover, the authors demonstrated that older humans with a gene expression profile normally associated with younger people tended to have a kidney in better condition for their age with a morphological appearance and a physiological state more similar to that of younger people. In contrast, a younger subject with gene expression profiles normally associated with a greater age, had a kidney in poorer condition for his age with a morphological appearance and physiological state more similar to that of much older people.

The results indicated that tissue gene expression profiles were able to be used to predict if humans have kidneys exhibiting an unusual state of wellness or, alternatively, abnormal degeneration for their respective chronological age. Finally, in two different types of human kidney tissue, from old and young human subjects the same gene varied suggesting that the same molecular differences between old and young cells would occur in all organs, and that there are common mechanisms of aging in all tissues.

Genes involved in the maintenance of normal physiological values of blood cholesterol (lipids), blood triglyceride lipids, blood Low Density Lipoprotein (LDL) and High Density Lipoprotein (HDL) and the LDL to HDL ratio, blood glucose, liver function, heart rate, protein, vitamin and mineral metabolism, immune system natural killer cell (NK) activity, immune system vitality and proportion of NK cells, immune cells (NK, B and T-cell counts) and immune T-cell helper/suppressor ratio, genes involved in apoptosis, cell adhesion, cell growth and maintenance, cytoskeletal organization, embryonal development, electron transport, endo-exophagocytosis, inflammation/immune response, metabolism of carbohydrates, fatty acids, lipids, nucleic acids, TCA cycle, protein folding, protein synthesis, protein ubiquitination, proteolysis, response to stress, signal transduction, transcription, or transport may be regarded as contributing especially to maintaining and promoting the state of wellness.

In terms of gene expression products, genes involved in the expression of IgF1r, Bcl2 antagonist, cyclooxygenase, especially genes involved in protein synthesis, turnover and modification, such as elF4A, 4E, 4gamma1, elF3subunit10, eukaryotic translation elongation factor 2, mitochondrial ribosomal protein L43, L27, ARF binding protein3, f-box only protein 9, DnaJ (Hsp40 homolog, Hsp1beta etc.) are vital to the maintenance and promotion of wellness.

Thus, the gene expression profile may be used as an indicator of the wellness state of an organism or of a rejuvenating effect. The higher capacity to maintain metabolic processes in balance and to maintain homeostasis in a young adult as compared to a corresponding older subject, may be reflected in its gene expression profile. Thus, the average gene expression profile of a young adult organism can be used as a reference for an optimal physiological state of wellness. Moreover, the state of wellness of an organism could be promoted by creating a younger environment for its cells by changing cellular signaling to a younger profile and as such restoring the capacity to maintain homeostasis and regenerative efficiency and thus also rejuvenating the organism. Genome-wide analysis of gene expression profiles enables the global assessment of the wellness state of a cell, tissue or organism. The comparison of the gene expression profile of an organism to that of a healthy younger adult organism can be used as a measure of the global wellness sate of the organism. A younger gene expression profile reflects a younger metabolic and signaling profile of a cell or organism, which will be more resistant to internal or external stimuli and, thus, maintains the organism in a better wellness state.

It has been found in accordance with the present invention that promotion of the wellness state of a mammal or a rejuvenating effect can be achieved by administering to said mammal an effective amount of resveratrol, a derivative, metabolite or analogue thereof.

The term “resveratrol, a derivative, metabolite or analogue thereof” as used herein comprises compounds encompassed by the general formula I

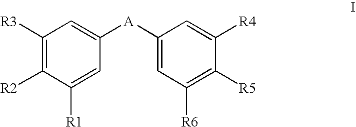

wherein A denotes a carbon-carbon single or double bond, the latter may be trans or cis, and R1, R2, R3, R4, R5 and R6, independently from each other denote hydrogen, hydroxy, etherified hydroxy or esterified hydroxy groups. Preferred are compounds I wherein A is a double bond (—CH═CH—).

While the carbon-carbon double bond denoted by the symbol A may be trans or cis, formula I above is understood to also include cis/trans mixtures. However, compounds of formula I wherein A is a trans carbon-carbon bond are preferred.

Etherified or esterified hydroxy groups may be derived from unsubstituted or substituted, straight- or branched-chain alkyl groups having 1 to 26 carbon atoms or from unsubstituted or substituted, straight- or branched-chain aliphatic, araliphatic or aromatic carboxylic acids having 1 to 26 carbon atoms. Etherified hydroxy groups may further be glycoside groups, and esterified hydroxy groups may further be glucuronide or sulfate groups. Examples of compounds of formula I wherein A is —CH═CH— are resveratrol (R1, R3 and R5=hydrogen, R2, R4 and R6=hydroxy); piceatannol (R3 and R5=hydrogen, R1, R2, R4 and R6=hydroxy), and rhapontigenin (R5=hydrogen, R1, R3, R4 and R6=hydroxy, and R2=methoxy). Examples of compounds of formula I wherein A is —CH₂—CH₂— are dihydroresveratrol (R1, R3 and R5=hydrogen; R2, R4 and R6=hydroxy), dihydropiceatannol (R3 and R5=hydrogen; R1, R2, R4 and R6=hydroxy) and tristin (R3 and R5=hydrogen; R2, R4 and R6=hydroxy and R1=methoxy). These compounds are all well-known and commercially available or can be obtained in accordance with methods well-known in the art.

For the purposes of the invention, resveratrol, a derivative, metabolite or analogue thereof may be of synthetic or of natural origin. In one preferred embodiment of the invention, resveratrol, particularly (trans)-resveratrol, of synthetic origin is used for the purposes of the invention. In another embodiment of the invention, resveratrol of natural origin is used, i.e., isolated from natural resveratrol sources, or as a resveratrol-containing extract from natural resveratrol sources such as grape seed extract or giant knotweed extract. Furthermore, resveratrol may be used for the purposes of the invention alone, i.e., as a single active component or in combination with one or more other active ingredients often used in nutritional supplemental formulations. Such other ingredients include, but are not restricted to, mineral salts; vitamins (e.g., vitamin E and C); carotenoids, such as β-carotene, lycopene, lutein or zeaxanthin; green tea catechins, such as epigallocatechin (EGCG); olive phenolics, such as hydroxytyrosol and oleuropein; Coenzyme Q10; genistein and PUFAs of all kinds, especially in the form of their esters, naturally occurring, in the form of extracts and concentrates or synthetically produced and in more or less pure form.

In accordance with the present invention, it has been found that the gene expression profile of a mammal whose diet is supplemented with resveratrol, a derivative, metabolite or analogue thereof is closer to the profile which is found in a healthy young adult mammal than to the profile which is found in a mammal having the same chronological age and whose diet was devoid of resveratrol. In other words, it has been found in accordance with the present invention that gene expression profiles in adult mammals can be changed towards conformity with expression profiles of younger adult mammals by administering to an adult mammal an effective amount of resveratrol, a derivative, metabolite or analogue thereof.

In view of the correlation between the gene expression profile and the physiological function of an organism, mammals treated with resveratrol, a derivative, metabolite or analogue thereof will be in a state of wellness more similar to that of a healthy younger organism than to the average wellness state of an organism of the same age whose diet has not been supplemented with resveratrol. Thus dietary resveratrol, a derivative, metabolite or analogue thereof promote the global wellness state of an organism by inducing physiological changes that will give the organism a younger phenotype. The promotion of wellness by dietary resveratrol in accordance with the invention improves mental fitness, enhances physical fitness, improves mobility and performance, promotes physical strength and mental strength, provides longevity and healthy aging. In other words, the typical result of the promotion of wellness is rejuvenation of or a rejuvenating effect on the mammal.

Thus, in one embodiment, the present invention is concerned with a method of promoting the wellness state of a mammal or rejuvenating a mammal, which comprises providing said mammal with an effective amount of resveratrol, a derivative, metabolite or analogue thereof.

The provision of the active compounds of the present invention is preferably via nutraceuticals.

Therefore, in yet another embodiment, the present invention is concerned with the use of resveratrol, a derivative, metabolite or analogue thereof, for the manufacture of a nutraceutical composition for promoting the wellness state of a mammal, and for changing gene expression profiles in older adult mammals towards conformity with expression profiles found in younger adult mammals which means rejuvenating said mammal.

The term <<nutraceutical>> as used herein refers to compositions for use in both the nutritional and pharmaceutical fields of application. Thus, a nutraceutical compositions can be a supplement to food and beverages, or a pharmaceutical formulations for enteral or parenteral application which may be solid formulations such as capsules or tablets, or liquid formulations, such as solutions or suspensions. The term <<food>> is used herein to also include animal feed. As will be evident from the foregoing, the term nutraceutical composition also comprises food and beverages containing the above-specified active ingredients as well as dosage unit compositions.

More specific embodiments of the present invention include, but are not limited to, the use of resveratrol, a derivative, metabolite or analogue thereof, for preventing imbalances in homeostasis by improving the cellular metabolism and performance and thus improving the body performance; maintaining body mobility; improving physical and mental fitness and strength; maintaining the regenerative and repair capacity of an organism, by enhancing the ability of that organism to maintain a young state; maintaining organ function, by avoiding physiological abnormality and/or biochemical irregularity-causing disorders; and promoting the ability of an organism to adapt to a changing environment.

The effects of resveratrol, a derivative, metabolite or analogue thereof on gene expression profiles can be determined by methods known per se, e.g., as described in more detail below.

### Animals and Dietary Manipulations

Male B6C3F, mice (6-7 weeks of age) were purchased from Harlan Sprague Dawley. Mice were housed singly and provided water ad libitum. The control group (OC, N=5) was fed 98 kcal/week of modified AIN-93M semi-purified diet (Bio-serv, Frenchtown, N.J.), which provides approximately 15% fewer calories than the average ad libitum dietary intake. The treatment group (RES, N=5) was fed the same caloric intake as controls, but were supplemented with resveratrol 50 mg/kg diet (w/w) (3,4′,5-Trihydroxy-trans-stilbene; Sigma) from 14 months of age. Animals (OC and RES) were sacrificed at 30 months of age. Young animals were sacrificed at 5 months of age (YC, N=5). Hearts from all abovementioned groups were collected, immediately frozen in liquid nitrogen and stored at −80° C. until analysis.

### Target RNA Preparation and High-Density Oligonucleotide Array Hybridizations

Total RNA was extracted from frozen tissue by using TRIZOL reagent (Life Technologies, Grand Island, N.Y.). Polyadenylate [poly(A)⁺] RNA was purified from total RNA with oligo-dT-linked oligotex resin (Qiagen, Valencia, Calif.). One μg of poly(A)⁺ RNA was converted into double-stranded cDNA (ds-cDNA) by using the SuperScript Choice System (Life Technologies), with an oligo-dT primer containing a T7 RNA polymerase promoter (Genset, La Jolla, Calif.). Ds-cDNA was extracted with phenol-chloroform-isoamyl alcohol and precipitated with pellet paint co-precipitant (Novagen, Madison, Wis.). Biotin-labeled RNA was synthesized in vitro using the BioArray High Yield RNA Transcript Labeling Kit (Enzo, Farmingdale, N.Y.). The biotin-labeled antisense cRNA was then purified using the RNeasy affinity column (Qiagen) and fragmented randomly. The hybridization cocktail (200 μl) containing 10 μg of fragmented cRNA was injected into the mouse Genome 430 2.0 DNA microarray (Affymetrix, Santa Clara, Calif.). After hybridization, the gene chips were washed and stained in a fluidic station (Model 800101, Affymetrix) with a signal amplification protocol using antibody. DNA chips were scanned at a resolution of 3 μm, twice, using a Hewlett-Packard GeneArray Scanner (Model 900154, Affymetrix) and the averaged images were used for further analysis.

### Data Analysis

Gene expression data were obtained using the Affymetrix Mouse Genome 430 2.0 array containing 45,101 probe sets. All steps, as detailed below, were performed using the most recent version of probe set (Mouse Genome 430.2.0 array probe set annotations available from Affymetrix at 23 Aug. 2005). Signal intensity was determined using Affymetrix's GeneChip Operating Software version 1.3. Data analysis was performed using Genedata Expressionist® Pro, version 2.0.

### Step 1 Data Acquisition and Quality Control

All data was imported into Genedata Expressionist® Pro. Refiner for quality control, using Diagnose with reference module. Samples that showed a high degree of variability from other similarly treated biological replicates were removed from the data set and excluded from further analysis. Two chips (one in the YC group, one in the RES group) were eliminated due to abnormal data distribution patterns. 18 chips were further analysed using Genedata Expressionist® Pro. Analyst.

Present and marginal signals were selected for further processing using the Affymetrix quality value of 0.065 (threshold for marginal expression). All data was normalized to the median of each chip.

In the further analyses, chips derived from the same treatment groups were grouped together and, for each probe, the group average was set as the median of the group. Only signals where at least 3 out of 5 chips (or 3 out of 4) were present/marginal were used for further selection.

### Step 2 Delete Ambiguous Probe Sets

According to Affymetrix's “Data Analysis Fundamentals” manual (http://www.affymetrix.com/support/downloads/manuals/data_analysis_fundamentals_manual.pdf, probe sets that contain the text “_x_at” or “_s_at” do not confidently query a single gene, thus they were filtered from the data set. In addition, probe sets not querying well-characterized genes were eliminated from further analysis. Examples include probe sets representing cDNA sequences, expressed sequence tags (ESTs), RIKEN cDNA sequences, DNA segments or hypothetical proteins.

### Step 3 Statistical Analysis

1) Genes Changed with Age

Genes which changed with age (ageing markers) were selected by comparing YC and OC groups. To determine if there was a change in the expression of a gene with resveratrol, comparisons were made between OC and RES mice, respectively. Significantly changed genes were selected using the n-fold test, with change >1.25-fold or consistently present in one group but absent in other groups, in combination with the two-sample test, where significance was tested at P<0.01 (both the t-test and Welch test were used to calculate the statistical significance).

2) Genes Unchanged with Age

The genes which were unchanged with age (non-ageing markers) were selected by comparing YC and OC groups, first filtered with a variance <0.05, then further selected with fold of change <1.25-fold, plus 2-group test, where significance was tested at p>0.01 (i.e. significantly unchanged between YC and OC groups). Similarly, significantly-changed genes were determined using either fold of change (>1.25-fold) or consistently present in one group but absent in other groups. Two-tailed t-test and Welch test were both used to calculate the statistical significance, where P<0.01 was considered as a significant change.

### Step 4 Classification Using Supervised Learning

Classification is a complex task which is divided into two phases, supervised learning and a test phase. The goal of classification is to predict an output variable (in this case, age) given an individual's input data. The supervised learning phase involves the application of an algorithm to training data, with the goal of learning rules by which the output variable can be predicted, while the test phase constitutes predictions being made for novel individuals, applying the rules collected in the learning phase. It is also possible to make predictions based on supplied training data.

For the classification analysis, supervised learning using Support Vector Machine (SVM) was performed based on supplied training data, “Young (YC)” and “Old (OC)” groups, to find rules for predicting the output variable “Age”. A cleaned probe set (see above) was employed. Results from the resveratrol group were then analysed using the Classification function for prediction of the output variable, “Age”. All 4 chips were classified as Young. A numeric value of classification output ranging from +1 to −1 was assigned to each chip, where +1 indicates a perfect match for a specific category, and −1 indicates a mismatch to the highest extent.

| | | Age | | |
|---|---|---|---|---|
| | | (Young = 1, | | |
| Group | Experiment | Od = −1) | Average of g | STEV |
| YC | DSh-yc2 | 1 | 0.9998575 | 0.000202711 |
| | DSh-yc3 | 1 | | |
| | DSh-yc4 | 0.99986 | | |
| | DSh-yc5 | 0.99957 | | |
| OC | DSh-oc1 | −1 | −1.023992 | 0.053670106 |
| | DSh-oc2 | −0.99996 | | |
| | DSh-oc3 | −1 | | |
| | DSh-oc4 | −1.12 | | |
| | DSh-oc5 | −1 | | |
| RES | DSh-Re1 | 0.51896 | 0.34855 | 0.140348421 |
| | DSh-Re2 | 0.25584 | | |
| | DSh-Re5 | 0.21368 | | |
| | DSh-Re7 | 0.40572 | | |

Table I: This table contains the numerical values of the classifier outputs from each chip; average (mean) and standard deviation are calculated for each group. The resveratrol group had a mean classification output of 0.34855, which is clearly much closer to the value calculated for the young group, than that of the old group.

The genome-wide gene expression in heart tissue in groups of young, old and resveratrol-fed mice was monitored using the mouse Affymetrix gene array. Among the 26,000 probe sets available on the chip, 1285 genes were significantly changed between the groups of young mice and old mice (at least 1.25-fold, where p<0.01). Among them, 501 genes were up- and 724 were down-regulated in the group of old mice. Within the 1285 genes, the expression of 585 genes in the resveratrol treated group were changed in the direction of the group of younger adult mice (45.5%, see table II), indicating a significant effect of resveratrol in maintaining the heart in a healthy and young state To summarize the scope of the resveratrol effect, in the 1023 above mentioned genes changed by resveratrol treatment, 342 resembled or exceeded the expression level of the young group (58.1%); 62 genes were at a level of 80-90% compared to the young group and 53 genes reached a level of 70-80%. Therefore, in spite of being of the same biological age as the mice in the group of old animals, old animals fed a resveratrol-supplemented diet showed a gene profile surprisingly close to that of the young animals.

TABLE II
Gene expression levels in resveratrol-fed old mice

| | number of genes |
|---|---|
| | changed by |
| Effect* | resveratrol |
| >100% | 340 |
| 90-100% | 62 |
| 80-90% | 52 |
| 70-80% | 48 |
| 60-70% | 39 |
| <60% | 44 |
| Sum | 585 |

Effect*: Gene expression levels in resveratrol-fed old mice expressed as percentage of gene expression levels observed in untreated young mice.

In Annex 1 (Table II.1) all 585 genes of Table II are identified specifically. It is well documented in the literature that the ageing process is accompanied by a decreased function of protein synthesis and protein folding, which leads to an imbalance of protein turnover, especially in muscle. Exercise has been shown to increase muscle protein synthesis and mitochondrial function in the elderly. In addition, protein folding and protein modification is also affected by ageing, as a consequence of physiological and pathological changes. Many diseases, such as Alzheimer's and Parkinson's disease, are associated with abnormal protein modification. In the old animal's group, significant reductions in many genes related to protein synthesis and protein folding (see table I) were observed. In contrast, in old animals fed a resveratrol-supplemented diet, many of these genes were upregulated to or close to the level found in young animals, e.g. genes involved in protein synthesis such as eukaryotic translation initiation factors 4A1, 2, 3, 4E, 4g1. Similarly, genes involved in protein modification, such as f-box only protein 9, homocysteine-inducible, endoplasmic reticulum stress-inducible, ubiquitin-like domain member 1 and ubiquitin specific protease 3 were all significantly down-regulated in the group of old animals, while these effects were reversed in the resveratrol group. (see Table III).

In conclusion, the present study showed that the gene expression profile of an adult mammal treated orally with resveratrol is closer to a healthy younger adult mammal than to a mammal having the same chronological age. This younger gene expression profile in resveratrol treated animals promotes the state of wellness in the animals and rejuvenates the animals.

TABLE III
Protein-synthesis, -turnover, -folding and -modification related genes.

| | | | | | | Gene Ontology |
|---|---|---|---|---|---|---|
| | Median | Median | Median | % of res | Genebank | Biological |
| Description | YC | OC | RES | effect | ID | Process |
| eukaryotic translation initiation | 71114 | 42480 | 71501 | 101.3515 | BI656407 | protein |
| factor 4A1 | | | | | | biosynthesis |
| eukaryotic translation initiation | 16118 | 4558 | 10650 | 52.69896 | NM_012010 | protein |
| factor 2, subunit 3, structural | | | | | | biosynthesis |
| gene X-linked | | | | | | |
| eukaryotic translation | 240011 | 355971 | 165689 | 164.0928 | BC007152 | protein |
| elongation factor 2 | | | | | | biosynthesis |
| eukaryotic translation initiation | 57542 | 83004 | 55723 | 107.144 | AW701127 | protein |
| factor 3, subunit 10 (theta) | | | | | | biosynthesis |
| suppressor of initiator codon | 186835 | 270767 | 203121 | 80.5962 | BI693609 | protein |
| mutations, related sequence 1 | | | | | | biosynthesis: |
| (S. cerevisiae) | | | | | | translational |
| | | | | | | initiation |
| eukaryotic translation initiation | 16080 | 10740 | 14990 | 79.58801 | AI449084 | protein |
| factor 4E like 3 | | | | | | biosynthesis: |
| | | | | | | translational |
| | | | | | | initiation: |
| | | | | | | regulation of |
| | | | | | | protein |
| | | | | | | biosynthesis: |
| | | | | | | regulation of |
| | | | | | | translation |
| Hbs1-like (S. cerevisiae) | 11040 | 3532 | 9115 | 74.36068 | AK012856 | protein |
| | | | | | | biosynthesis: |
| | | | | | | translational |
| | | | | | | elongation |
| eukaryotic translation initiation | 89231 | 131755 | 73196 | 137.7081 | BF227830 | protein |
| factor 4, gamma 1 | | | | | | biosynthesis: |
| | | | | | | regulation of |
| | | | | | | protein |
| | | | | | | biosynthesis: |
| | | | | | | regulation of |
| | | | | | | translation: |
| | | | | | | regulation of |
| | | | | | | translational |
| | | | | | | initiation |
| glutamyl-prolyl-tRNA | 14710 | 29050 | 16720 | 85.98326 | BM238943 | protein |
| synthetase | | | | | | biosynthesis: |
| | | | | | | tRNA |
| | | | | | | aminoacylation for |
| | | | | | | protein translation: |
| | | | | | | glutamyl-tRNA |
| | | | | | | aminoacylation: |
| | | | | | | prolyl-tRNA |
| | | | | | | aminoacylation |
| mitochondrial ribosomal protein | 29160 | 11990 | 25890 | 80.95515 | AK021196 | protein |
| L9 | | | | | | biosynthesis |
| eukaryotic translation initiation | 133319 | 94566 | 133932 | 101.5818 | BB406487 | protein |
| factor 4E | | | | | | biosynthesis: |
| | | | | | | translational |
| | | | | | | initiation: |
| | | | | | | regulation of |
| | | | | | | protein |
| | | | | | | biosynthesis: |
| | | | | | | regulation of |
| | | | | | | translation |
| eukaryotic translation initiation | 41881 | 32738 | 45897 | 143.9243 | BB406487 | protein |
| factor 4E | | | | | | biosynthesis: |
| | | | | | | translational |
| | | | | | | initiation: |
| | | | | | | regulation of |
| | | | | | | protein |
| | | | | | | biosynthesis: |
| | | | | | | regulation of |
| | | | | | | translation |
| ubiquitin protein ligase E3 | 7688 | 2342 | 7571 | 97.81145 | BQ173927 | protein |
| component n-recognin 1 | | | | | | biosynthesis: |
| | | | | | | ubiquitin- |
| | | | | | | dependent protein |
| | | | | | | catabolism: |
| | | | | | | ubiquitin- |
| | | | | | | dependent protein |
| | | | | | | catabolism: |
| | | | | | | ubiquitin cycle |
| ribonuclease P2 | 23520 | 16950 | 23730 | 103.1963 | BG069849 | protein |
| | | | | | | biosynthesis: |
| | | | | | | tRNA processing |
| mitochondrial ribosomal protein | 60787 | 45205 | 61436 | 104.1651 | NM_053164 | protein |
| L43 | | | | | | biosynthesis |
| mitochondrial ribosomal protein | 77648 | 60618 | 91619 | 182.0376 | NM_053161 | protein |
| L27 | | | | | | biosynthesis |
| basic leucine zipper and W2 | 21900 | 7072 | 19500 | 83.81441 | AV144956 | regulation of |
| domains 1 | | | | | | translational |
| | | | | | | initiation |
| basic leucine zipper and W2 | 37458 | 29355 | 38679 | 115.0685 | AV144956 | regulation of |
| domains 1 | | | | | | translational |
| | | | | | | initiation |
| basic leucine zipper and W2 | 22760 | 15014 | 25745 | 138.536 | AV144956 | regulation of |
| domains 1 | | | | | | translational |
| | | | | | | initiation |
| DnaJ (Hsp40) homolog, | 25270 | 13854 | 20850 | 61.28241 | NM_021422 | protein folding |
| subfamily A, member 4 | | | | | | |
| DnaJ (Hsp40) homolog, | 34315 | 53571 | 29661 | 124.1691 | NM_020266 | protein folding |
| subfamily B, member 10 | | | | | | |
| DnaJ (Hsp40) homolog, | 21719 | 28290 | 17969 | 157.0689 | AI664344 | protein folding |
| subfamily B, member 5 | | | | | | |
| FK506 binding protein 7 | 24440 | 14640 | 33928 | 196.8163 | NM_010222 | protein folding |
| sarcolemma associated protein | 11797 | 7201 | 11160 | 86.14012 | NM_032008 | protein folding |
| calreticulin 3 | 9676 | 15010 | 8927 | 114.042 | AI324734 | protein folding |
| calnexin | 30200 | 18950 | 30204 | 100.0356 | BI653492 | protein folding |
| tumor rejection antigen gp96 | 55362 | 30536 | 53900 | 94.11101 | BE995678 | protein folding |
| Mus musculus transcribed | 37670 | 27670 | 38260 | 105.9 | AK004331 | protein folding: |
| sequences | | | | | | transport: |
| | | | | | | neurotransmitter |
| | | | | | | transport |
| DnaJ (Hsp40) homolog, | 35780 | 19520 | 30553 | 67.85363 | AK004575 | protein folding: |
| subfamily A, member 3 | | | | | | apoptosis: small |
| | | | | | | GTPase mediated |
| | | | | | | signal |
| | | | | | | transduction |
| Bcl2-associated athanogene 3 | 81895 | 127237 | 67398 | 131.9726 | NM_013863 | protein folding: |
| | | | | | | apoptosis: anti- |
| | | | | | | apoptosis: |
| | | | | | | regulation of |
| | | | | | | apoptosis: |
| | | | | | | negative |
| | | | | | | regulation of |
| | | | | | | apoptosis |
| heat shock protein 1, beta | 193270 | 272021 | 150912 | 153.7873 | BI154147 | protein folding: |
| | | | | | | response to |
| | | | | | | unfolded protein: |
| | | | | | | response to heat |
| golgi associated, gamma | 7471 | 10170 | 5532 | 171.8414 | BB501734 | protein complex |
| adaptin ear containing, ARF | | | | | | assembly: |
| binding protein 3 | | | | | | transport: |
| | | | | | | intracellular |
| | | | | | | protein transport: |
| | | | | | | intra-Golgi |
| | | | | | | transport: |
| | | | | | | protein transport |
| expressed sequence AI663987 | 19610 | 28610 | 12520 | 178.7778 | BB764994 | protein |
| | | | | | | modification |
| ubiquitin-conjugating enzyme | 45944 | 27305 | 40250 | 69.45115 | AK009276 | protein |
| E2D 3 (UBC4/5 homolog, | | | | | | modification: |
| yeast) | | | | | | ubiquitin- |
| | | | | | | dependent protein |
| | | | | | | catabolism: |
| | | | | | | ubiquitin cycle: |
| | | | | | | proteasomal |
| | | | | | | ubiquitin- |
| | | | | | | dependent protein |
| | | | | | | catabolism |
| neural precursor cell | 105341 | 65626 | 86633 | 52.89437 | BG073415 | protein |
| expressed, developmentally | | | | | | modification: |
| down-regulted gene 4 | | | | | | ubiquitin cycle |
| homocysteine-inducible, | 84354 | 60092 | 90964 | 127.2443 | AI835088 | protein |
| endoplasmic reticulum stress- | | | | | | modification: |
| inducible, ubiquitin-like domain | | | | | | response to stress: |
| member 1 | | | | | | response to |
| | | | | | | unfolded protein |
| homocysteine-inducible, | 100279 | 77635 | 107786 | 133.1523 | NM_022331 | protein |
| endoplasmic reticulum stress- | | | | | | modification: |
| inducible, ubiquitin-like domain | | | | | | response to stress: |
| member 1 | | | | | | response to |
| | | | | | | unfolded protein |
| ubiquitin specific protease 3 | 8753 | 6879 | 9725 | 151.8677 | BM936366 | protein |
| | | | | | | modification: |
| | | | | | | ubiquitin- |
| | | | | | | dependent protein |
| | | | | | | catabolism: |
| | | | | | | ubiquitin cycle: |
| | | | | | | protein |
| | | | | | | deubiquitination |
| f-box only protein 9 | 8000 | 3513 | 6555 | 67.79585 | AK018482 | protein |
| | | | | | | modification: |
| | | | | | | ubiquitin cycle: |
| | | | | | | protein |
| | | | | | | ubiquitination |
| f-box only protein 9 | 40058 | 25420 | 48649 | 158.6897 | NM_023605 | protein |
| | | | | | | modification // |
| | | | | | | inferred from |
| | | | | | | sequence or |
| | | | | | | structural |
| | | | | | | similarity: ubiquitin |
| | | | | | | cycle: protein |
| | | | | | | ubiquitination |
| gene trap locus 6 | 45441 | 60873 | 40130 | 134.4155 | BG923744 | protein |
| | | | | | | modification: |
| | | | | | | ubiquitin cycle |
| casein kinase II, alpha 1 | 6243 | 8116 | 5588 | 134.9706 | BB283759 | protein amino acid |
| polypeptide | | | | | | phosphorylation: |
| | | | | | | Wnt receptor |
| | | | | | | signaling pathway |
| serum/glucocorticoid regulated | 59987 | 42724 | 72667 | 173.4519 | NM_011361 | protein amino acid |
| kinase | | | | | | phosphorylation: |
| | | | | | | apoptosis: |
| | | | | | | response to DNA |
| | | | | | | damage stimulus |
| protein kinase, interferon | 11780 | 8692 | 13990 | 171.5674 | NM_011871 | protein amino acid |
| inducible double stranded RNA | | | | | | phosphorylation: |
| dependent activator | | | | | | response to stress |
| mitogen activated protein | 11444 | 18511 | 9718 | 124.4234 | U11548 | protein amino acid |
| kinase kinase kinase 4 | | | | | | phosphorylation |
| dystrophia myotonica kinase, | 134121 | 289851 | 62794 | 145.8017 | AW108486 | protein amino acid |
| B15 | | | | | | phosphorylation |
| transient receptor potential | 17042 | 8788 | 19508 | 129.8764 | AV320241 | protein amino acid |
| cation channel, subfamily M, | | | | | | phosphorylation: |
| member 7 | | | | | | transport: ion |
| | | | | | | transport: cation |
| | | | | | | transport: |
| | | | | | | calcium ion |
| | | | | | | transport |
| large tumor suppressor 2 | 8425 | 4618 | 6754 | 56.10717 | BE986745 | protein amino acid |
| | | | | | | phosphorylation: |
| | | | | | | cell cycle: |
| | | | | | | mitosis: |
| | | | | | | negative |
| | | | | | | regulation of cell |
| | | | | | | cycle: cell |
| | | | | | | division |
| PTEN induced putative kinase 1 | 142460 | 196243 | 116921 | 147.4853 | AF316872 | protein amino acid |
| | | | | | | phosphorylation |
| Mus musculus similar to heart | 39310 | 58530 | 21847 | 190.8585 | AY044451 | protein amino acid |
| alpha-kinase (LOC381181), | | | | | | phosphorylation |
| mRNA | | | | | | |
| serine/threonine kinase 17b | 8122 | 4957 | 8039 | 97.37757 | AV173139 | protein amino acid |
| (apoptosis-inducing) | | | | | | phosphorylation: |
| serine/threonine kinase 17b | 9718 | 5996 | 12069 | 163.165 | AV173139 | protein amino acid |
| (apoptosis-inducing) | | | | | | phosphorylation: |
| tousled-like kinase 1 | 5085 | 2664 | 4589 | 79.5126 | BM244995 | protein amino acid |
| | | | | | | phosphorylation: |
| | | | | | | response to DNA |
| | | | | | | damage stimulus: |
| | | | | | | cell cycle: |
| | | | | | | chromatin |
| | | | | | | modification |
| G protein-coupled receptor | 32682 | 57251 | 37493 | 80.41841 | BC019379 | protein amino acid |
| kinase 5 | | | | | | phosphorylation: |
| | | | | | | signal |
| | | | | | | transduction: G- |
| | | | | | | protein coupled |
| | | | | | | receptor protein |
| | | | | | | signaling pathway |
| microtubule associated | 62173 | 89788 | 45140 | 161.6802 | NM_008641 | protein amino acid |
| serine/threonine kinase 2 | | | | | | phosphorylation: |
| | | | | | | intracellular |
| | | | | | | signaling cascade |
| expressed sequence | 22660 | 33000 | 19940 | 126.3056 | NM_054085 | protein amino acid |
| AW319487 | | | | | | phosphorylation: |
| | | | | | | heart |
| | | | | | | development |
| protein kinase inhibitor, alpha | 53479 | 20510 | 40073 | 59.33756 | AK010212 | negative |
| | | | | | | regulation of |
| | | | | | | protein kinase |
| | | | | | | activity |
| protein phosphatase 1B, | 43020 | 69113 | 52488 | 63.71441 | AJ271833 | protein amino acid |
| magnesium dependent, beta | | | | | | dephosphorylation/ |
| isoform | | | | | | |
| dual specificity phosphatase 6 | 13390 | 8439 | 13740 | 107.0693 | NM_026268 | protein amino acid |
| | | | | | | dephosphorylation |
| No description found | 7973 | 12590 | 9370 | 69.74226 | BG976607 | protein amino acid |
| | | | | | | dephosphorylation: |
| | | | | | | muscle |
| | | | | | | maintenance: |
| | | | | | | phospholipid |
| | | | | | | dephosphorylation |
| myotubularin related protein 1 | 15202 | 11220 | 14340 | 78.35259 | BB381813 | protein amino acid |
| | | | | | | dephosphorylation: |
| | | | | | | phospholipid |
| | | | | | | dephosphorylation |
| protein tyrosine phosphatase, | 9982 | 14910 | 4135 | 218.6485 | AW987375 | protein amino acid |
| non-receptor type 21 | | | | | | dephosphorylation: |
| | | | | | | proteolysis and |
| | | | | | | peptidolysis |
| acid phosphatase 1, soluble | 21840 | 16710 | 34652 | 349.7466 | AW554436 | protein amino acid |
| | | | | | | dephosphorylation |
| protein tyrosine phosphatase- | 111425 | 57783 | 98966 | 76.7738 | BB014781 | protein amino acid |
| like (proline instead of catalytic | | | | | | dephosphorylation |
| arginine), member a | | | | | | |
| myotubularin related protein 6 | 17166 | 10940 | 16336 | 86.66881 | BC020019 | protein amino acid |
| | | | | | | dephosphorylation |
| protein tyrosine phosphatase | 235270 | 113062 | 200548 | 71.58778 | AW495875 | protein amino acid |
| 4a2 | | | | | | dephosphorylation |
| protein tyrosine phosphatase | 110805 | 164010 | 52165 | 210.2152 | AK014601 | protein amino acid |
| 4a3 | | | | | | dephosphorylation |
| ADP-ribosyltransferase 1 | 84854 | 46341 | 87939 | 108.0103 | NM_009710 | protein amino acid |
| | | | | | | ADP-ribosylation |
| isoprenylcysteine carboxyl | 7362 | 9194 | 6260 | 160.1528 | BF462080 | C-terminal protein |
| methyltransferase | | | | | | amino acid |
| | | | | | | methylation: |
| | | | | | | protein |
| | | | | | | localization |
| sialyltransferase 10 (alpha-2,3- | 17290 | 12060 | 16650 | 87.76291 | NM_018784 | protein amino acid |
| sialyltransferase VI) | | | | | | glycosylation |
| O-linked N-acetylglucosamine | 3713 | 6564 | 2989 | 125.3946 | BG065325 | O-linked |
| (GlcNAc) transferase (UDP-N- | | | | | | glycosylation |
| acetylglucosamine:polypeptide- | | | | | | |
| N-acetylglucosaminyl | | | | | | |
| transferase) | | | | | | |
| puromycin-sensitive | 28602 | 36550 | 19697 | 212.0408 | AK010446 | proteolysis and |
| aminopeptidase | | | | | | peptidolysis |
| cathepsin Z | 41394 | 31066 | 43097 | 116.4892 | NM_022325 | proteolysis and |
| | | | | | | peptidolysis |
| insulin degrading enzyme | 26080 | 41145 | 25035 | 106.9366 | AV027702 | proteolysis and |
| | | | | | | peptidolysis |
| membrane metallo | 7864 | 4476 | 8513 | 119.1558 | AV174022 | proteolysis and |
| endopeptidase [BLAST] | | | | | | peptidolysis |
| furin (paired basic amino acid | 13220 | 18650 | 8279 | 190.9945 | NM_011046 | proteolysis and |
| cleaving enzyme) | | | | | | peptidolysis |
| ring finger protein 13 | 26420 | 10480 | 33170 | 142.3463 | NM_011883 | proteolysis and |
| | | | | | | peptidolysis: |
| | | | | | | protein |
| | | | | | | ubiquitination |
| cathepsin B | 211604 | 138105 | 194089 | 76.16974 | M14222 | proteolysis and |
| | | | | | | peptidolysis: |
| | | | | | | protein targeting |
| transferrin receptor | 137622 | 26991 | 86773 | 54.0373 | AK011596 | proteolysis and |
| | | | | | | peptidolysis: iron |
| | | | | | | ion homeostasis: |
| | | | | | | endocytosis |
| methionyl aminopeptidase 1 | 11830 | 4926 | 10496 | 80.67787 | BG064851 | proteolysis and |
| | | | | | | peptidolysis |
| lipocalin 7 | 24779 | 37955 | 14280 | 179.6828 | BC005738 | proteolysis and |
| | | | | | | peptidolysis: |
| | | | | | | transport |
| dynein, cytoplasmic, heavy | 42912 | 67491 | 30053 | 152.317 | NM_030238 | proteolysis and |
| chain 1 | | | | | | peptidolysis: |
| | | | | | | microtubule- |
| | | | | | | based movement |
| proteasome (prosome, | 138180 | 102392 | 139883 | 104.7586 | NM_008944 | ubiquitin- |
| macropain) subunit, alpha type 2 | | | | | | dependent protein |
| | | | | | | catabolism |
| cylindromatosis (turban tumor | 8404 | 12270 | 8255 | 103.8541 | AK013508 | ubiquitin- |
| syndrome) | | | | | | dependent protein |
| | | | | | | catabolism: |
| | | | | | | ubiquitin cycle |
| ubiquitin specific protease 10 | 17575 | 23930 | 12090 | 186.31 | NM_009462 | ubiquitin- |
| | | | | | | dependent protein |
| | | | | | | catabolism: |
| | | | | | | ubiquitin cycle |
| F-box protein 30 | 8641 | 3802 | 6641 | 58.66915 | AK006369 | ubiquitin cycle |
| F-box only protein 32 | 34704 | 8833 | 35761 | 104.0857 | AF441120 | ubiquitin cycle: |
| | | | | | | transport |
| F-box only protein 25 | 12506 | 7558 | 11530 | 80.27486 | NM_025785 | ubiquitin cycle: |
| | | | | | | transport |
| Stam binding protein | 10200 | 7600 | 13590 | 230.3846 | AK019907 | ubiquitin cycle: |
| | | | | | | anti-apoptosis: |
| | | | | | | signal |
| | | | | | | transduction |
| cullin 3 | 24780 | 15280 | 22604 | 77.09474 | AV273804 | ubiquitin cycle: |
| | | | | | | cell cycle |
| anaphase-promoting complex | 38273 | 17970 | 33166 | 74.84608 | AK003821 | ubiquitin cycle: |
| subunit 5 | | | | | | cell cycle: |
| | | | | | | mitosis: cell |
| | | | | | | division |
| synaptojanin 2 binding protein | 36777 | 18550 | 40710 | 121.5779 | AK008254 | protein targeting: |
| | | | | | | cytoplasm |
| | | | | | | organization and |
| | | | | | | biogenesis: |
| | | | | | | intracellular |
| | | | | | | signaling cascade: |
| | | | | | | Rho protein |
| | | | | | | signal |
| | | | | | | transduction: |
| | | | | | | regulation of |
| | | | | | | endocytosis |
| ribosome binding protein 1 | 46822 | 63729 | 37971 | 152.3511 | AK019964 | protein targeting |
| | | | | | | transport: protein |
| | | | | | | transport |

For the purposes of the invention, the dosage requirements for resveratrol, a derivative, metabolite or analogue are not narrowly critical. Amounts of up to about 30 mg/kg body weight per day or even higher, depending of the nature of the mammal concerned and its condition and requirements may be administered. Thus, for a human adult (weighing about 70 kg) the dosage may be up to about 2000 mg/day. In a particular embodiment of the invention, the dosage for a human adult (weighing about 70 kg) is up to about 500 mg/day, especially up to about 500 mg/day. Suitably, the dosage is no less than 0.5 mg. In a particular embodiment of the invention, the dosage for a human adult (weighing about 70 kg) is no less than 2 mg., especially is no less than 5 mg. If administered in a food or beverage the amount of resveratrol, a derivative, metabolite or analogue thereof contained therein is suitably no less than about 0.2 mg per serving. In another embodiment of the invention such amount is no less than 2 mg per serving. On the other side, resveratrol, a derivative, metabolite or analogue thereof may be administered in a food or beverage in an amount of up to 100, 200 or 500 mg per serving. If resveratrol, a derivative, metabolite or analogue thereof is adminstered as a pharmaceutical formulation such formulation may contain up to about 100, 200 or 500 mg per solid dosage unit, e.g., per capsule or tablet, or up to about 2000 mg per daily dose of a liquid formulation. If resveratrol is used as an extract from natural sources the above dosage figures refer to the amount of pure resveratrol contained in the extract.

The term “serving” as used herein denotes an amount of food or beverage normally ingested by a human adult with a meal at a time and may range, e.g., from about 100 g to about 500 g.

For the purposes of the present invention resveratrol, a derivative, metabolite or analogue thereof may be administered as a nutritional supplement, e.g., as an additive to a multi-vitamin preparations comprising vitamins and minerals which are essential for the maintenance of normal metabolic function. Resveratrol, a derivative, metabolite or analogue thereof may be adminstered also as a pharmaceutical composition, preferably for enteral application, which may be solid or liquid galenical formulation. Examples of solid galenical formulations are tablets, capsules (e.g. hard or soft shell gelatin capsules), pills, sachets, powders, granules and the like which contain the active ingredient together with conventional galenical carriers. Any conventional carrier material can be utilized. The carrier material can be organic or inorganic inert carrier material suitable for oral administration. Suitable carriers include water, gelatin, gum arabic, lactose, starch, magnesium stearate, talc, vegetable oils, and the like. Additionally, additives such as flavouring agents, preservatives, stabilizers, emulsifying agents, buffers and the like may be added in accordance with accepted practices of pharmaceutical compounding. While the individual active ingredients are suitably administered in a single composition they may also be administered in individual dosage units.

The following Examples illustrate the invention further

Pharmaceutical compositions may be prepared by conventional formulation procedures

### EXAMPLE 1

### Soft Gelatin Capsule

Soft gelatin capsules are prepared by conventional procedures containing as active ingredient 30 mg of resveratrol per capsule.

### EXAMPLE 2

### Hard Gelatin Capsule

Hard gelatin capsules are prepared by conventional procedures containing as active ingredient 20 mg of resveratrol per capsule.

### EXAMPLE 3

### Tablet

Tablets are prepared by conventional procedures containing as active ingredient 10 mg of resveratrol per tablet, and as excipients microcrystalline cellulose, silicone dioxide (SiO2), magnesium stearate, crospovidone NF (which is a disintegration agent) ad 200 mg.

### EXAMPLE 4

Food items may be prepared by conventional procedures containing resveratrol in an amount of 0.2 mg to 200 mg per serving. Examples of such food items are soft drinks, bread, cookies, yogurt, ice cream, and sweets.

For example an orange-Lemon juice drink, containing 10% juice and resveratrol is prepared from the following ingredients:

| Ingredients | [g] |
|---|---|
| Sugar syrup 64° Brix | 156.2 |
| Sodium benzoate | 0.2 |
| Ascorbic acid, fine powder | 0.2 |
| Citric acid 50% w/w | 5.0 |
| Pectin solution 2% w/w | 10.0 |
| Resveratrol | 0.02 |
| Juice compound* | 30.0 |
| Water to | 250.0 |

### Preparation

Dissolve sodium benzoate in water whilst stirring
Continue stirring and add sugar syrup, ascorbic acid, citric acid, pectin solution, juice compound, one after the other. Do not use a high speed mixer
Dilute the bottling syrup with (carbonated) water to one liter of beverage

| *Ingredients Juice compound | [g] |
|---|---|
| Orange juice concentrate 65° Brix | 483.3 |
| Lemon Juice Concentrate 45° Brix | 173.3 |
| Oily orange flavour | 5.0 |
| β-Carotene 10% CWS as 10% stocksolution | 10.0 |
| Deionized water | 328.4 |

### Preparation of Juice Compound

Add the deionized water to the juice concentrates, stir gently and allow the juice concentrates to hydrate.
Add the oily flavour and β-Carotene 10% CWS stocksolution and pre-emulsify in a rotor-stator-homogenizer.
Homogenize in a high-pressure homogenizer at 200 bar.

### Addition of β-Carotene 10% CWS

β-Carotene 10% CWS should be added to the juice compound as a 1-10% stocksolution in deionized water

### EXAMPLE 5

A reconvalescent 70 year old person weighing 55 kg is administered resveratrol at a dosage regimen of 20 mg per day for a duration of 2 months.

### EXAMPLE 6

A person aged 55 years weighing 70 kg intending to participate in a sporting event is administered 30 mg of resveratrol per day for 3 months before said event.

### EXAMPLE 7

A person aged 60 years weighing 75 kg complaining of frequent episodes of tiredness and general lack of motivation is administered 50 mg of resveratrol per day for 3 months.

TABLE II.1
Annex 1:

| Effect of | | |
|---|---|---|
| resveratrol on | | |
| changeing | | |
| gene | | |
| expression | | |
| from old to | | |
| young profile | Name | Description |
| >100% | 1460028_at | Mus musculus transcribed sequences |
| | 1434652_at | Cdc42 binding protein kinase beta |
| | 1450974_at | tissue inhibitor of metalloproteinase 4 |
| | 1434560_at | KIAA1037 protein |
| | 1421441_at | angiopoietin |
| | 1416542_at | PHD finger protein 1 |
| | 1450191_a_at | SRY-box containing gene 13 |
| | 1419295_at | cAMP responsive element binding protein 3-like 1 |
| | 1448690_at | potassium channel, subfamily K, member 1 |
| | 1448424_at | frizzled-related protein |
| | 1450720_at | acid phosphatase 1, soluble |
| | 1422799_at | HLA-B associated transcript 2 |
| | 1448825_at | pyruvate dehydrogenase kinase, isoenzyme 2 |
| | 1420727_a_at | trimethyllysine hydroxylase, epsilon |
| | 1417628_at | suppressor of Ty 6 homolog (S. cerevisiae) |
| | 1433890_a_at | HLA-B-associated transcript 3 |
| | 1451012_a_at | cold shock domain protein A |
| | 1448703_at | LSM8 homolog, U6 small nuclear RNA associated (S. cerevisiae) |
| | 1431962_a_at | Stam binding protein |
| | 1451422_at | myosin XVIIIa |
| | 1434516_at | hypothetical protein 5730458D16 |
| | 1421197_a_at | apoptotic chromatin condensation inducer in the nucleus |
| | 1420991_at | ankyrin repeat domain 1 (cardiac muscle) |
| | 1419054_a_at | protein tyrosine phosphatase, non-receptor type 21 |
| | 1425341_at | potassium channel, subfamily K, member 3 |
| | 1421654_a_at | lamin A |
| | 1422521_at | dynactin 1 |
| | 1417386_at | puromycin-sensitive aminopeptidase |
| | 1416532_at | expressed sequence AI481500 |
| | 1418181_at | protein tyrosine phosphatase 4a3 |
| | 1416066_at | CD9 antigen |
| | 1449117_at | Jun proto-oncogene related gene d1 |
| | 1427052_at | acetyl-Coenzyme A carboxylase beta |
| | 1418589_a_at | myeloid leukemia factor 1 |
| | 1451168_a_at | Rho GDP dissociation inhibitor (GDI) alpha |
| | 1416374_at | adaptor-related protein complex 3, mu 1 subunit |
| | 1416803_at | FK506 binding protein 7 |
| | 1428382_at | Mus musculus transcribed sequence with moderate similarity to protein |
| | | ref: NP_033237.1 (M. musculus) SWI/SNF related, matrix associated, |
| | | actin dependent regulator of chromatin, subfamily c, member . . . |
| | 1455285_at | solute carrier family 31, member 1 |
| | 1425927_a_at | activating transcription factor 5 |
| | 1418518_at | furin (paired basic amino acid cleaving enzyme) |
| | 1452478_at | Mus musculus similar to heart alpha-kinase (LOC381181), mRNA |
| | 1424359_at | Mus musculus transcribed sequence with weak similarity to protein |
| | | ref: NP_061137.1 (H. sapiens) sphingomyelin phosphodiesterase 3, |
| | | neutral membrane (neutral sphingomyelinase II) [Homo sapiens] |
| | 1448121_at | WW domain binding protein 2 |
| | 1422522_at | fragile X mental retardation gene 2, autosomal homolog |
| | 1448230_at | ubiquitin specific protease 10 |
| | 1415690_at | mitochondrial ribosomal protein L27 |
| | 1417557_at | UBX domain containing 1 |
| | 1417109_at | lipocalin 7 |
| | 1427039_at | epsin 1 |
| | 1450958_at | transmembrane 4 superfamily member 1 |
| | 1448691_at | expressed sequence AI663987 |
| | 1434893_at | ATPase, Na+/K+ transporting, alpha 2 polypeptide [BLAST] |
| | 1452690_at | KH-type splicing regulatory protein |
| | 1427079_at | microtubule-associated protein, RP/EB family, member 3 |
| | 1451350_a_at | leptin receptor gene-related protein |
| | 1450777_at | 5′-3′ exoribonuclease 2 |
| | 1460302_at | thrombospondin 1 |
| | 1429139_at | zinc finger, A20 domain containing 1 |
| | 1440870_at | PR domain containing 16 |
| | 1454868_at | expressed sequence AV028368 |
| | 1416041_at | serum/glucocorticoid regulated kinase |
| | 1417964_at | adaptor-related protein complex 3, delta subunit |
| | 1455404_at | junctophilin 2 |
| | 1438008_at | golgi associated, gamma adaptin ear containing, ARF binding protein 3 |
| | 1416652_at | asporin |
| | 1448923_at | protein kinase, interferon inducible double stranded RNA dependent |
| | | activator |
| | 1421743_a_at | poly(rC) binding protein 2 |
| | 1422414_a_at | calmodulin 3 |
| | 1455539_at | glycosylphosphatidylinositol specific phospholipase D1 [BLAST] |
| | 1451586_at | testis enhanced gene transcript |
| | 1455725_a_at | H3 histone, family 3B |
| | 1415713_a_at | DEAD (Asp-Glu-Ala-Asp) box polypeptide 24 |
| | 1455101_at | Mus musculus, clone IMAGE: 2647821, mRNA |
| | 1422811_at | solute carrier family 27 (fatty acid transporter), member 1 |
| | 1421072_at | Iroquois related homeobox 5 (Drosophila) |
| | 1418502_a_at | oxidation resistance 1 |
| | 1424736_at | eukaryotic translation elongation factor 2 |
| | 1418621_at | RAB2, member RAS oncogene family |
| | 1423452_at | serine/threonine kinase 17b (apoptosis-inducing) |
| | 1426353_at | signal transducer and activator of transcription 6 |
| | 1452298_a_at | Mus musculus similar to myosin homolog, brain - mouse (LOC383411), |
| | | mRNA |
| | 1417324_at | microtubule associated serine/threonine kinase 2 |
| | 1418835_at | pleckstrin homology-like domain, family A, member 1 |
| | 1448189_a_at | flightless I homolog (Drosophila) |
| | 1417480_at | f-box only protein 9 |
| | 1417423_at | glutamate receptor, ionotropic, N-methyl D-asparate-associated protein 1 |
| | | (glutamate binding) |
| | 1454675_at | thyroid hormone receptor alpha |
| | 1439530_a_at | Mus musculus similar to RelA-associated inhibitor (Inhibitor of ASPP |
| | | protein) (Protein iASPP) (PPP1R13B-like protein) (LOC243869), mRNA |
| | 1426731_at | desmin |
| | 1455870_at | Mus musculus transcribed sequence with weak similarity to protein |
| | | sp: Q9Y2D5 (H. sapiens) AKA2_HUMAN A-kinase anchor protein 2 |
| | | (Protein kinase A anchoring protein 2) (PRKA2) |
| | 1448151_at | ELAV (embryonic lethal, abnormal vision, Drosophila)-like 1 (Hu antigen |
| | | R) |
| | 1416590_a_at | RAB34, member of RAS oncogene family |
| | 1421962_at | DnaJ (Hsp40) homolog, subfamily B, member 5 |
| | 1421861_at | calsyntenin 1 |
| | 1437729_at | ribosomal protein L27a |
| | 1416364_at | heat shock protein 1, beta |
| | 1451295_a_at | chromodomain helicase DNA binding protein 4 |
| | 1430536_a_at | Mus musculus transcribed sequence with strong similarity to protein |
| | | prf: 2208314A (H. sapiens) 2208314A ERH gene [Homo sapiens] [BLAST] |
| | 1418706_at | solute carrier family 38, member 3 |
| | 1450634_at | ATPase, H+ transporting, V1 subunit A, isoform 1 |
| | 1452767_at | ribosome binding protein 1 |
| | 1416648_at | dynein, cytoplasmic, heavy chain 1 |
| | 1431054_at | LSM6 homolog, U6 small nuclear RNA associated (S. cerevisiae) |
| | 1425507_at | ADP-ribosylation factor related protein 1 |
| | 1425022_at | ubiquitin specific protease 3 |
| | 1424164_at | mitochondrial ribosomal protein L50 |
| | 1420896_at | synaptosomal-associated protein 23 |
| | 1423126_at | ATPase, Na+/K+ transporting, beta 3 polypeptide |
| | 1418468_at | annexin A11 |
| | 1427245_at | ADP-ribosylation factor GTPase activating protein 1 |
| | 1435658_at | solute carrier family 27 (fatty acid transporter), member 1 |
| | 1418128_at | adenylate cyclase 6 |
| | 1452066_a_at | Nedd4 family interacting protein 2 |
| | 1418648_at | EGL nine homolog 3 (C. elegans) |
| | 1420899_at | RAB18, member RAS oncogene family |
| | 1415741_at | TPA regulated locus |
| | 1460199_a_at | platelet-activating factor acetylhydrolase, isoform 1b, beta1 subunit |
| | 1453124_at | transportin 3 |
| | 1417968_a_at | methyl-CpG binding domain protein 1 |
| | 1451148_at | PTEN induced putative kinase 1 |
| | 1426440_at | dehydrogenase/reductase (SDR family) member 7 |
| | 1417982_at | insulin induced gene 2 |
| | 1417502_at | transmembrane 4 superfamily member 2 |
| | 1419220_at | cardiomyopathy associated 1 |
| | 1434944_at | dystrophia myotonica kinase, B15 |
| | 1425281_a_at | delta sleep inducing peptide, immunoreactor |
| | 1454848_at | protein phosphatase 1, regulatory (inhibitor) subunit 12C [BLAST] |
| | 1452843_at | interleukin 6 signal transducer |
| | 1423220_at | eukaryotic translation initiation factor 4E |
| | 1448938_at | replication protein A3 |
| | 1417947_at | proliferating cell nuclear antigen |
| | 1418663_at | multiple PDZ domain protein |
| | 1450008_a_at | catenin beta |
| | 1426613_a_at | U2 small nuclear ribonucleoprotein B |
| | 1453129_a_at | regulator of G-protein signaling 12 |
| | 1435472_at | kringle containing transmembrane protein |
| | 1455702_at | WD repeat domain 22 |
| | 1427874_at | zinc finger protein 313 |
| | 1420620_a_at | ring finger protein 13 |
| | 1454811_a_at | tumor differentially expressed 2 |
| | 1427045_at | synaptopodin |
| | 1429186_a_at | cytidine and dCMP deaminase domain containing 1 |
| | 1435664_at | zinc finger protein 397 |
| | 1433558_at | disabled homolog 2 (Drosophila) interacting protein |
| | 1416113_at | FK506 binding protein 8 |
| | 1416698_a_at | CDC28 protein kinase 1 |
| | 1418595_at | plasma membrane associated protein, S3-12 |
| | 1423607_at | lumican |
| | 1437892_at | Mus musculus transcribed sequences |
| | 1445186_at | stanniocalcin 2 |
| | 1420834_at | vesicle-associated membrane protein 2 |
| | 1449116_a_at | deoxythymidylate kinase |
| | 1460174_at | dexamethasone-induced transcript |
| | 1423040_at | basic leucine zipper and W2 domains 1 |
| | 1449334_at | tissue inhibitor of metalloproteinase 3 |
| | 1427036_a_at | eukaryotic translation initiation factor 4, gamma 1 |
| | 1418524_at | pericentriolar material 1 |
| | 1454974_at | syntaxin 8 |
| | 1444089_at | spectrin beta 2 |
| | 1416167_at | peroxiredoxin 4 |
| | 1418509_at | carbonyl reductase 2 |
| | 1434930_at | two pore channel 1 |
| | 1448277_at | polymerase (DNA directed), delta 2, regulatory subunit |
| | 1423474_at | topoisomerase (DNA) I |
| | 1451249_at | DNA segment, Chr 8, ERATO Doi 812, expressed |
| | 1425977_a_at | serine/threonine kinase 2 |
| | 1421468_at | potassium inwardly-rectifying channel, subfamily J, member 3 |
| | 1455182_at | kinesin family member 1B |
| | 1419034_at | casein kinase II, alpha 1 polypeptide |
| | 1450048_a_at | vacuolar protein sorting 33B (yeast) [BLAST] |
| | 1423898_a_at | gene trap locus 6 |
| | 1433811_at | myeloid/lymphoid or mixed lineage-leukemia translocation to 6 homolog |
| | | (Drosophila) |
| | 1455506_at | Mus musculus transcribed sequences |
| | 1419665_a_at | nuclear protein 1 |
| | 1448185_at | homocysteine-inducible, endoplasmic reticulum stress-inducible, ubiquitin- |
| | | like domain member 1 |
| | 1450899_at | neural precursor cell expressed, developmentally down-regulated gene 1 |
| | 1448307_at | Down syndrome critical region homolog 2 (human) |
| | 1423845_at | expressed sequence AI481750 |
| | 1422452_at | Bcl2-associated athanogene 3 |
| | 1433904_at | BCL2-antagonist/killer 1 |
| | 1427006_at | guanine nucleotide releasing factor 2 |
| | 1420920_a_at | ADP-ribosylation factor 1 |
| | 1416647_at | branched chain ketoacid dehydrogenase E1, alpha polypeptide |
| | 1454837_at | ceroid-lipofuscinosis, neuronal 6 |
| | 1417149_at | procollagen-proline, 2-oxoglutarate 4-dioxygenase (proline 4- |
| | | hydroxylase), alpha II polypeptide |
| | 1448119_at | 2,3-bisphosphoglycerate mutase |
| | 1436960_at | bromodomain containing 3 |
| | 1416800_at | transient receptor potential cation channel, subfamily M, member 7 |
| | 1456930_at | hypothetical protein 9530003A05 |
| | 1424622_at | heat shock factor 1 |
| | 1416194_at | cytochrome P450, family 4, subfamily b, polypeptide 1 |
| | 1450917_at | myomesin 2 |
| | 1455518_at | Mus musculus transcribed sequences |
| | 1454708_at | actin-binding LIM protein 1 |
| | 1448618_at | major vault protein |
| | 1424776_a_at | solute carrier family 25, member 28 |
| | 1448390_a_at | dehydrogenase/reductase (SDR family) member 3 |
| | 1435626_a_at | homocysteine-inducible, endoplasmic reticulum stress-inducible, ubiquitin- |
| | | like domain member 1 |
| | 1419440_at | ring finger protein 30 |
| | 1415838_at | tumor differentially expressed 2 |
| | 1450724_at | down-regulated by Ctnnb1, a |
| | 1451825_a_at | coatomer protein complex, subunit zeta 1 |
| | 1449573_at | expressed sequence AW319487 |
| | 1456341_a_at | basic transcription element binding protein 1 |
| | 1434815_a_at | expressed sequence AI874665 |
| | 1450957_a_at | sequestosome 1 |
| | 1423332_at | syndecan binding protein |
| | 1451229_at | histone deacetylase 11 |
| | 1424111_at | insulin-like growth factor 2 receptor |
| | 1424269_a_at | Mus musculus similar to myosin light chain, alkali, nonmuscle |
| | | (LOC383643), mRNA |
| | 1423793_at | DNA segment, Chr 2, ERATO Doi 391, expressed |
| | 1426486_at | UBX domain containing 2 |
| | 1421450_a_at | mitogen activated protein kinase kinase kinase 4 |
| | 1425280_at | transmembrane channel-like gene family 4 |
| | 1448657_a_at | DnaJ (Hsp40) homolog, subfamily B, member 10 |
| | 1421054_at | exportin 4 |
| | 1425956_a_at | cytidine and dCMP deaminase domain containing 1 |
| | 1449635_at | DNA segment, Chr 19, Wayne State University 55, expressed |
| | 1438545_at | solute carrier family 25 (mitochondrial carrier; adenine nucleotide |
| | | translocator), member 5 |
| | 1451519_at | ring finger protein 2 |
| | 1448467_a_at | tangerin |
| | 1429348_at | sema domain, immunoglobulin domain (Ig), short basic domain, secreted, |
| | | (semaphorin) 3C |
| | 1436173_at | deleted in liver cancer 1 |
| | 1418098_at | adenylate cyclase 4 |
| | 1420900_a_at | RAB18, member RAS oncogene family |
| | 1431507_a_at | synaptojanin 2 binding protein |
| | 1460337_at | SH3-domain kinase binding protein 1 |
| | 1448448_a_at | choline kinase-like |
| | 1422569_at | YY1 transcription factor |
| | 1416091_at | microtubule-associated protein 4 |
| | 1434924_at | PHD finger protein 2 |
| | 1460246_at | methyl CpG binding protein 2 |
| | 1434416_a_at | small optic lobes homolog (Drosophila) |
| | 1450759_at | bone morphogenetic protein 6 |
| | 1416958_at | nuclear receptor subfamily 1, group D, member 2 |
| | 1455961_at | membrane metallo endopeptidase [BLAST] |
| | 1418527_a_at | neural-salient serine/arginine-rich |
| | 1415966_a_at | NADH dehydrogenase (ubiquinone) flavoprotein 1 |
| | 1454928_at | hypothetical protein E130307D12 |
| | 1460565_at | solute carrier family 41, member 1 |
| | 1434189_at | stromal antigen 1 |
| | 1460556_at | DNA Segment, Chr 15 Massachusetts Institute of Technology 260 |
| | 1435679_at | optineurin |
| | 1434834_at | Nck, Ash and phospholipase C binding protein |
| | 1421139_a_at | zinc finger protein 386 (Kruppel-like) |
| | 1450699_at | selenium binding protein 1 |
| | 1416593_at | glutaredoxin 1 (thioltransferase) |
| | 1416595_at | mitochondrial ribosomal protein S22 |
| | 1417868_a_at | cathepsin Z |
| | 1455247_at | angiomotin-like 1 |
| | 1437875_at | bicaudal D homolog 2 (Drosophila) |
| | 1423345_at | degenerative spermatocyte homolog (Drosophila) |
| | 1426403_at | ARP1 actin-related protein 1 homolog B (yeast) |
| | 1454696_at | guanine nucleotide binding protein, beta 1 |
| | 1422538_at | exotoses (multiple)-like 2 |
| | 1423041_a_at | basic leucine zipper and W2 domains 1 |
| | 1448688_at | podocalyxin-like |
| | 1448503_at | myeloid cell leukemia sequence 1 |
| | 1431255_at | calreticulin 3 |
| | 1418454_at | microfibrillar associated protein 5 |
| | 1423063_at | DNA methyltransferase 3A |
| | 1415882_at | growth hormone inducible transmembrane protein |
| | 1416320_at | SEC22 vesicle trafficking protein-like 2 (S. cerevisiae) |
| | 1450890_a_at | abl-interactor 1 |
| | 1455136_at | ATPase, Na+/K+ transporting, alpha 2 polypeptide |
| | 1418117_at | NADH dehydrogenase (ubiquinone) Fe—S protein 4 |
| | 1427432_a_at | Mus musculus transcribed sequences |
| | 1424109_a_at | glyoxalase 1 |
| | 1425745_a_at | transforming, acidic coiled-coil containing protein 2 |
| | 1424669_at | zinc finger, FYVE domain containing 21 |
| | 1435730_at | Mus musculus transcribed sequences |
| | 1415735_at | damage specific DNA binding protein 1 |
| | 1439478_at | mitochondrial acyl-CoA thioesterase 1 [BLAST] |
| | 1453406_a_at | RAB28, member RAS oncogene family |
| | 1433461_at | splicing factor 3b, subunit 2 |
| | 1449349_at | nudix (nucleoside diphosphate linked moiety X)-type motif 1 |
| | 1425702_a_at | ectonucleotide pyrophosphatase/phosphodiesterase 5 |
| | 1424538_at | ubiquitin-like 4 |
| | 1416852_a_at | expressed sequence AU040320 |
| | 1460287_at | tissue inhibitor of metalloproteinase 2 |
| | 1420889_at | holocytochrome c synthetase |
| | 1417933_at | keratin complex 2, basic, gene 8 |
| | 1418701_at | armadillo repeat gene deleted in velo-cardio-facial syndrome |
| | 1436014_a_at | RUN and SH3 domain containing 1 |
| | 1422375_a_at | ADP-ribosyltransferase 1 |
| | 1415989_at | vascular cell adhesion molecule 1 |
| | 1415763_a_at | Mus musculus transcribed sequence |
| | 1434254_at | hypothetical protein E430025L19 |
| | 1426465_at | discs, large homolog-associated protein 4 (Drosophila) |
| | 1423198_a_at | expressed sequence AW011752 |
| | 1418401_a_at | dual specificity phosphatase 16 |
| | 1416659_at | eukaryotic translation initiation factor 3, subunit 10 (theta) |
| | 1415834_at | dual specificity phosphatase 6 |
| | 1456623_at | tropomyosin 1, alpha |
| | 1435140_at | insulin degrading enzyme |
| | 1419041_at | DNA segment, Chr 8, Wayne State University 49, expressed |
| | 1424389_at | nucleoporin like 1 |
| | 1419365_at | adaptor-related protein complex 3, sigma 2 subunit |
| | 1432016_a_at | isocitrate dehydrogenase 3 (NAD+) alpha [BLAST] |
| | 1425196_a_at | histidine triad nucleotide binding protein 2 |
| | 1424141_at | HECT domain containing 1 |
| | 1434302_at | No description found |
| | 1435652_a_at | guanine nucleotide binding protein, alpha inhibiting 2 |
| | 1428892_at | Mus musculus transcribed sequences |
| | 1415864_at | 2,3-bisphosphoglycerate mutase |
| | 1448206_at | proteasome (prosome, macropain) subunit, alpha type 2 |
| | 1435893_at | very low density lipoprotein receptor |
| | 1421871_at | SH3-binding domain glutamic acid-rich protein like |
| | 1438673_at | solute carrier family 4, sodium bicarbonate cotransporter, member 7 |
| | 1415681_at | mitochondrial ribosomal protein L43 |
| | 1448747_at | F-box only protein 32 |
| | 1454603_a_at | CCR4-NOT transcription complex, subunit 2 |
| | 1429201_at | cylindromatosis (turban tumor syndrome) |
| | 1448309_at | adaptor-related protein complex 3, mu 1 subunit |
| | 1451457_at | sterol-C5-desaturase (fungal ERG3, delta-5-desaturase) homolog (S. cerevisae) |
| | 1428454_at | breast carcinoma amplified sequence 3 |
| | 1420427_a_at | DEAH (Asp-Glu-Ala-His) box polypeptide 32 |
| | 1423373_at | ribonuclease P2 |
| | 1416923_a_at | BCL2/adenovirus E1B 19 kDa-interacting protein 3-like |
| | 1428563_at | DEAD (Asp-Glu-Ala-Asp) box polypeptide 10 |
| | 1439800_at | Mus musculus transcribed sequences |
| | 1436839_at | coactosin-like 1 (Dictyostelium) |
| | 1418627_at | glutamate-cysteine ligase, modifier subunit |
| | 1436348_at | Mus musculus transcribed sequences |
| | 1455048_at | immunoglobulin superfamily, member 2 |
| | 1418030_at | solute carrier organic anion transporter family, member 3a1 |
| | 1448330_at | glutathione S-transferase, mu 1 |
| | 1437513_a_at | tumor differentially expressed 2 |
| | 1430999_a_at | short coiled-coil protein |
| | 1450908_at | eukaryotic translation initiation factor 4E |
| | 1419259_at | Ras suppressor protein 1 |
| | 1449574_a_at | cell division cycle 42 homolog (S. cerevisiae) |
| | 1430980_a_at | eukaryotic translation initiation factor 4A1 |
| | 1436420_a_at | importin 4 |
| | 1435213_at | NHL repeat containing 1 |
| | 1431746_a_at | ubiquitin-activating enzyme E1C [BLAST] |
| | 1417673_at | growth factor receptor bound protein 14 |
| | 1422842_at | 5′-3′ exoribonuclease 2 |
| | 1422845_at | calnexin |
| 90-100% | 1418843_at | solute carrier family 30 (zinc transporter), member 4 |
| | 1448625_at | golgi autoantigen, golgin subfamily a, 2 |
| | 1448700_at | G0/G1 switch gene 2 |
| | 1437879_at | Mus musculus similar to potassium channel regulator 1 (LOC380959), |
| | | mRNA |
| | 1455393_at | ceruloplasmin |
| | 1448568_a_at | solute carrier family 20, member 1 |
| | 1418692_at | RAB8A, member RAS oncogene family |
| | 1435032_at | golgi autoantigen, golgin subfamily b, macrogolgin 1 |
| | 1419300_at | FMS-like tyrosine kinase 1 |
| | 1452044_at | actin related protein 2/3 complex, subunit 5-like |
| | 1460213_at | golgi autoantigen, golgin subfamily a, 4 |
| | 1430421_a_at | hypothetical protein MGC18837 |
| | 1425542_a_at | protein phosphatase 2, regulatory subunit B (B56), gamma isoform |
| | 1456080_a_at | tumor differentially expressed 1 |
| | 1433539_at | COMM domain containing 3 |
| | 1420971_at | ubiquitin protein ligase E3 component n-recognin 1 |
| | 1421102_a_at | vesicle-associated membrane protein 3 |
| | 1432042_a_at | smu-1 suppressor of mec-8 and unc-52 homolog (C. elegans) |
| | 1424167_a_at | phosphomannomutase 1 |
| | 1450997_at | serine/threonine kinase 17b (apoptosis-inducing) |
| | 1428367_at | N-deacetylase/N-sulfotransferase (heparan glucosaminyl) 1 |
| | 1451336_at | lectin, galactose binding, soluble 4 |
| | 1420607_at | RNA binding motif protein 18 |
| | 1452670_at | myosin, light polypeptide 9, regulatory |
| | 1431012_a_at | peroxisomal delta3, delta2-enoyl-Coenzyme A isomerase |
| | 1455107_at | Mus musculus transcribed sequence with weak similarity to protein |
| | | pir: I58401 (M. musculus) I58401 protein-tyrosine kinase (EC 2.7.1.112) |
| | | JAK3 —mouse |
| | 1435265_at | Mus musculus transcribed sequence with strong similarity to protein |
| | | sp: P00722 (E. coli) BGAL_ECOLI Beta-galactosidase (Lactase) |
| | 1416794_at | ADP-ribosylation factor-like 6 interacting protein 2 |
| | 1420731_a_at | cysteine and glycine-rich protein 2 |
| | 1423627_at | NAD(P)H dehydrogenase, quinone 1 |
| | 1421116_a_at | reticulon 4 |
| | 1424314_at | PRP3 pre-mRNA processing factor 3 homolog (yeast) |
| | 1452985_at | uveal autoantigen with coiled-coil domains and ankyrin repeats |
| | 1418768_at | optic atrophy 1 homolog (human) |
| | 1449065_at | cytosolic acyl-CoA thioesterase 1 |
| | 1454699_at | sestrin 1 |
| | 1455156_at | striatin, calmodulin binding protein |
| | 1436809_a_at | spindlin |
| | 1438040_a_at | tumor rejection antigen gp96 |
| | 1416525_at | speckle-type POZ protein |
| | 1423482_at | uroporphyrinogen III synthase |
| | 1456813_at | Mus musculus transcribed sequences |
| | 1451124_at | superoxide dismutase 1, soluble |
| | 1450010_at | hydroxysteroid (17-beta) dehydrogenase 12 |
| | 1428372_at | suppression of tumorigenicity 5 |
| | 1449928_at | t-complex-associated-testis-expressed 1-like |
| | 1457701_at | Mus musculus transcribed sequence with strong similarity to protein |
| | | sp: P00722 (E. coli) BGAL_ECOLI Beta-galactosidase (Lactase) |
| | 1419543_a_at | Mus musculus similar to splicing factor, arginine/serine-rich (transformer 2 |
| | | Drosophila homolog) 10 (LOC229280), mRNA |
| | 1448791_at | sorting nexin 5 |
| | 1416973_at | sperm specific antigen 1 |
| | 1453740_a_at | cyclin L2 |
| | 1460320_at | beclin 1 (coiled-coil, myosin-like BCL2-interacting protein) |
| | 1450839_at | DNA segment, human D4S114 |
| | 1417539_at | solute carrier family 35 (CMP-sialic acid transporter), member 1 |
| | 1449503_at | karyopherin (importin) alpha 1 |
| | 1428471_at | sorbin and SH3 domain containing 1 |
| | 1434044_at | hypothetical protein C130032F08 |
| | 1428295_at | synaptopodin 2-like |
| | 1428645_at | guanine nucleotide binding protein, alpha inhibiting 3 |
| | 1431431_a_at | nitrogen fixation gene 1 (S. cerevisiae) |
| | 1417764_at | signal sequence receptor, alpha |
| | 1418406_at | phosphodiesterase 8A |
| 80-90% | 1422879_at | Mus musculus transcribed sequences |
| | 1420959_at | aspartate-beta-hydroxylase |
| | 1452156_a_at | nischarin |
| | 1417668_at | reticulon 4 interacting protein 1 |
| | 1428061_at | histidine aminotransferase 1 |
| | 1448553_at | myosin, heavy polypeptide 6, cardiac muscle, alpha |
| | 1416706_at | ribulose-5-phosphate-3-epimerase |
| | 1427490_at | ATP-binding cassette, sub-family B (MDR/TAP), member 7 |
| | 1423368_at | lysosomal-associated protein transmembrane 4A |
| | 1434937_at | pam, highwire, rpm 1 [BLAST] |
| | 1426983_at | formin binding protein 1 |
| | 1417734_at | neighbor of A-kinase anchoring protein 95 |
| | 1419662_at | osteoglycin |
| | 1451726_at | myotubularin related protein 6 |
| | 1423108_at | solute carrier family 25 (mitochondrial carnitine/acylcarnitine translocase), |
| | | member 20 |
| | 1433751_at | solute carrier family 39 (zinc transporter), member 10 |
| | 1421215_a_at | sarcolemma associated protein |
| | 1452157_at | glutamyl-prolyl-tRNA synthetase |
| | 1423051_at | heterogeneous nuclear ribonucleoprotein U |
| | 1429830_a_at | CD59a antigen |
| | 1435518_at | RAS related protein 1b |
| | 1448363_at | yes-associated protein |
| | 1448538_a_at | DNA segment, Chr 4, Wayne State University 53, expressed |
| | 1418231_at | LIM and senescent cell antigen-like domains 1 |
| | 1417220_at | fumarylacetoacetate hydrolase |
| | 1452879_at | synaptopodin 2 |
| | 1458302_at | Mus musculus transcribed sequences |
| | 1417442_a_at | peroxisomal biogenesis factor 3 |
| | 1450846_at | basic leucine zipper and W2 domains 1 |
| | 1419639_at | ephrin B2 |
| | 1428587_at | DNA segment, Chr 7, ERATO Doi 743, expressed |
| | 1437149_at | solute carrier family 6 (neurotransmitter transporter, taurine), member 6 |
| | | [BLAST] |
| | 1434268_at | adenosine deaminase, RNA-specific |
| | 1427165_at | interleukin 13 receptor, alpha 1 |
| | 1423720_a_at | SAR1a gene homolog 1 (S. cerevisiae) |
| | 1434537_at | solute carrier organic anion transporter family, member 3a1 |
| | 1437637_at | putative homeodomain transcription factor 2 |
| | 1451047_at | integral membrane protein 2A |
| | 1430533_a_at | catenin beta |
| | 1450853_at | transducin-like enhancer of split 4, homolog of Drosophila E(spl) |
| | 1450416_at | chromobox homolog 5 (Drosophila HP1a) |
| | 1415704_a_at | carnitine deficiency-associated gene expressed in ventricle 3 |
| | 1430976_a_at | mitochondrial ribosomal protein L9 |
| | 1426837_at | methionyl aminopeptidase 1 |
| | 1423799_at | suppressor of initiator codon mutations, related sequence 1 (S. cerevisiae) |
| | 1422834_at | potassium voltage-gated channel, Shal-related family, member 2 |
| | 1448997_at | pleckstrin homology, Sec7 and coiled-coil domains 1 |
| | 1430332_a_at | beta-glucuronidase |
| | 1449514_at | G protein-coupled receptor kinase 5 |
| | 1417535_at | F-box only protein 25 |
| | 1416988_at | mutS homolog 2 (E. coli) |
| | 1423042_at | DEAD/H (Asp-Glu-Ala-Asp/His) box polypeptide 3, X-linked |
| 70-80% | 1421333_a_at | myoneurin |
| | 1421985_a_at | eukaryotic translation initiation factor 4E like 3 |
| | 1456407_a_at | tousled-like kinase 1 |
| | 1454843_at | phosphoribosyl pyrophosphate synthetase 2 |
| | 1454966_at | integrin alpha 8 |
| | 1438368_a_at | matrin 3 |
| | 1426824_at | proteasome (prosome, macropain) activator subunit 4 |
| | 1423413_at | N-myc downstream regulated 1 |
| | 1444112_at | Mus musculus LOC381492 (LOC381492), mRNA |
| | 1421880_at | myotubularin related protein 1 |
| | 1426313_at | brain and reproductive organ-expressed protein |
| | 1421854_at | fibrinogen-like protein 2 |
| | 1431345_a_at | TATA box binding protein (Tbp)-associated factor, RNA polymerase I, B |
| | 1434548_at | tumor differentially expressed 1 |
| | 1450016_at | cyclin G1 |
| | 1422795_at | cullin 3 |
| | 1456315_a_at | protein tyrosine phosphatase-like (proline instead of catalytic arginine), |
| | | member a |
| | 1425929_a_at | ring finger protein 14 |
| | 1448732_at | cathepsin B |
| | 1420542_at | open reading frame 28 |
| | 1431030_a_at | ring finger protein 14 |
| | 1449024_a_at | hexosaminidase A |
| | 1416950_at | tumor necrosis factor, alpha-induced protein 8 |
| | 1425140_at | lactamase, beta 2 |
| | 1427902_at | serine/arginine repetitive matrix 2 |
| | 1455173_at | G1 to phase transition 1 |
| | 1453307_a_at | anaphase-promoting complex subunit 5 |
| | 1450396_at | stromal antigen 2 |
| | 1453604_a_at | Hbs1-like (S. cerevisiae) |
| | 1451072_a_at | ring finger protein 4 [BLAST] |
| | 1425631_at | protein phosphatase 1, regulatory (inhibitor) subunit 3C |
| | 1416084_at | zinc finger protein 216 |
| | 1431592_a_at | SH3-domain kinase binding protein 1 |
| | 1434935_at | expressed sequence C79663 |
| | 1420806_at | fibroblast growth factor 16 |
| | 1419288_at | junction adhesion molecule 2 |
| | 1446812_at | Mus musculus transcribed sequence with weak similarity to protein |
| | | pir: S12207 (M. musculus) S12207 hypothetical protein (B2 element) - |
| | | mouse |
| | 1435129_at | protein tyrosine phosphatase 4a2 |
| | 1450462_at | corticotropin releasing hormone receptor 2 |
| | 1460367_at | high mobility group box transcription factor 1 |
| | 1420533_at | guanylate cyclase 1, soluble, alpha 3 |
| | 1439811_at | 5-methyltetrahydrofolate-homocysteine methyltransferase |
| | 1423734_at | RAS-related C3 botulinum substrate 1 |
| | 1439214_a_at | apoptosis inhibitor 5 |
| | 1424648_at | RAB, member of RAS oncogene family-like 4 |
| | 1421832_at | twisted gastrulation homolog 1 (Drosophila) |
| | 1422690_at | serine palmitoyltransferase, long chain base subunit 1 |
| | 1453960_a_at | capping protein (actin filament) muscle Z-line, beta |
| 60-70% | 1423652_at | HESB like domain containing 2 |
| | 1434278_at | No description found |
| | 1427548_a_at | chloride channel, nucleotide-sensitive, 1A |
| | 1434105_at | EPM2A (laforin) interacting protein 1 |
| | 1429859_a_at | ADP-ribosylation factor-like 2 binding protein |
| | 1423114_at | ubiquitin-conjugating enzyme E2D 3 (UBC4/5 homolog, yeast) |
| | 1417374_at | tubulin, alpha 4 |
| | 1416408_at | acyl-Coenzyme A oxidase 1, palmitoyl |
| | 1449137_at | pyruvate dehydrogenase E1 alpha 1 |
| | 1416499_a_at | dynactin 6 |
| | 1416368_at | glutathione S-transferase, alpha 4 |
| | 1452545_a_at | integrin beta 1 (fibronectin receptor beta) |
| | 1420629_a_at | DnaJ (Hsp40) homolog, subfamily A, member 3 |
| | 1432211_a_at | f-box only protein 9 |
| | 1424671_at | pleckstrin homology domain containing, family F (with FYVE domain) |
| | | member 1 |
| | 1425158_at | T-box 20 |
| | 1428021_at | methylcrotonoyl-Coenzyme A carboxylase 2 (beta) |
| | 1419257_at | transcription elongation factor A (SII) 1 |
| | 1434580_at | ectonucleotide pyrophosphatase/phosphodiesterase 4 |
| | 1450351_a_at | restin (Reed-Steinberg cell-expressed intermediate filament-associated |
| | | protein) |
| | 1423089_at | tropomodulin 3 |
| | 1456981_at | transmembrane channel-like gene family 7 |
| | 1417736_at | SMC6 structural maintenance of chromosomes 6-like 1 (yeast) |
| | 1452207_at | Cbp/p300-interacting transactivator, with Glu/Asp-rich carboxy-terminal |
| | | domain, 2 |
| | 1424126_at | aminolevulinic acid synthase 1 |
| | 1437901_a_at | vacuolar protein sorting 41 (yeast) |
| | 1452011_a_at | UDP-glucuronate decarboxylase 1 |
| | 1450017_at | cyclin G1 |
| | 1417350_at | pallidin |
| | 1426382_at | protein phosphatase 1B, magnesium dependent, beta isoform |
| | 1448525_a_at | BCL2/adenovirus E1B 19 kDa-interacting protein 3-like |
| | 1454774_at | expressed sequence AW610627 |
| | 1417497_at | ceruloplasmin |
| | 1436098_at | butyrylcholinesterase |
| | 1436590_at | protein phosphatase 1, regulatory (inhibitor) subunit 3B |
| | 1418435_at | makorin, ring finger protein, 1 |
| | 1418592_at | DnaJ (Hsp40) homolog, subfamily A, member 4 |
| | 1433576_at | methionine adenosyltransferase II, alpha |
| | 1422470_at | BCL2/adenovirus E1B 19 kDa-interacting protein 1, NIP3 |
| <60% | 1432488_a_at | splicing factor 3a, subunit 3, 60 kDa |
| | 1422090_a_at | 6-phosphofructo-2-kinase/fructose-2,6-biphosphatase 2 |
| | 1432344_a_at | amyloid beta (A4) precursor-like protein 2 |
| | 1420858_at | protein kinase inhibitor, alpha |
| | 1434008_at | Mus musculus, clone IMAGE: 1282676, mRNA |
| | 1453137_at | F-box protein 30 |
| | 1450006_at | nuclear receptor coactivator 4 |
| | 1420871_at | guanylate cyclase 1, soluble, beta 3 |
| | 1419088_at | tissue inhibitor of metalloproteinase 3 |
| | 1459238_at | Mus musculus transcribed sequences |
| | 1421828_at | karyopherin (importin) alpha 3 |
| | 1415801_at | gap junction membrane channel protein alpha 1 |
| | 1450385_at | karyopherin (importin) alpha 3 |
| | 1419678_at | large tumor suppressor 2 |
| | 1422772_at | core 1 UDP-galactose:N-acetylgalactosamine-alpha-R beta 1,3- |
| | | galactosyltransferase |
| | 1448364_at | cyclin G2 |
| | 1430519_a_at | CCR4-NOT transcription complex, subunit 7 |
| | 1431645_a_at | guanosine diphosphate (GDP) dissociation inhibitor 3 |
| | 1452661_at | transferrin receptor |
| | 1451871_a_at | growth hormone receptor |
| | 1429533_at | inner membrane protein, mitochondrial |
| | 1451109_a_at | neural precursor cell expressed, developmentally down-regulted gene 4 |
| | 1421895_at | eukaryotic translation initiation factor 2, subunit 3, structural gene X-linked |
| | 1425097_a_at | zinc finger protein 106 |
| | 1443755_at | No description found |
| | 1425533_a_at | staufen (RNA binding protein) homolog 2 (Drosophila) |
| | 1421239_at | interleukin 6 signal transducer |
| | 1456620_at | v-maf musculoaponeurotic fibrosarcoma oncogene family, protein B |
| | | (avian) |
| | 1451345_at | methylthioadenosine phosphorylase |
| | 1453818_a_at | hypothetical gene supported by AK078282; AK078855, BC052508 |
| | 1415748_a_at | dynactin 5 |
| | 1456398_at | expressed sequence AI316828 |
| | 1448183_a_at | hypoxia inducible factor 1, alpha subunit |
| | 1452433_at | No description found |
| | 1417729_at | myosin, heavy polypeptide 6, cardiac muscle, alpha |
| | 1419098_at | erythrocyte protein band 7.2 |
| | 1448541_at | kinesin 2 |
| | 1426519_at | procollagen-proline, 2-oxoglutarate 4-dioxygenase (proline 4- |
| | | hydroxylase), alpha 1 polypeptide |
| | 1427610_at | desmoplakin |
| | 1437863_at | butyrylcholinesterase |
| | 1426066_a_at | dystrobrevin alpha |
| | 1416601_a_at | Down syndrome critical region homolog 1 (human) |
| | 1448348_at | GPI-anchored membrane protein 1 |
| | 1449341_a_at | erythrocyte protein band 7.2 |

1. The use of resveratrol, a derivative, metabolite or analogue thereof for the manufacture of a nutraceutical composition for promoting the wellness state of a mammal.

2. The use of resveratrol, a derivative, metabolite or analogue thereof for promoting the wellness state of a mammal by providing via a nutraceutical composition an effective amount of such compound to a mammal.

3. The use of resveratrol, a derivative, metabolite or analogue thereof according to claim 1 wherein the promotion of the wellness state is achieved by changing gene expression profiles in older adult mammals towards conformity with expression profiles found in younger adult mammals.

4. The use as in claim 1 wherein the genes are those which are expressed differently in younger and in older healthy mammals.

5. The use as in claim 1 wherein the genes are those listed in Table II.1, preferably those expression of which is changed by at least 60%, 70%, 80%, 90%, 100% or more than 100%, most preferably those expression of which is changed by more than 100%.

6. The use as in claim 1 wherein the resveratrol derivative, metabolite or analogue thereof is of synthetic origin.

7. The use as in claim 1 wherein the resveratrol, derivative, metabolite or analogue thereof is a resveratrol-containing extract from natural resveratrol sources.

8. The use as in claim 1 wherein the resveratrol has been isolated from natural resveratrol sources.

9. The use as in claim 1 wherein the nutraceutical composition is a food additive, a food or a beverage.

10. The use as in claim 9 wherein the food or the beverage contains resveratrol, a derivative, metabolite or analogue thereof in an amount sufficient to provide no less than 0.2 mg per serving.

11. The use as in claim 9 wherein the food or the beverage contains resveratrol, a derivative, metabolite or analogue thereof in an amount sufficient to provide no less than 2 mg per serving.

12. The use as in claim 1 wherein the nutraceutical composition is a dosage unit composition.

13. The use as in claim 12 wherein the dosage unit contains no less than 0.5 mg of resveratrol, a derivative, metabolite or analogue thereof.

14. The use as in claim 12 wherein the dosage unit contains no less than 5 mg of resveratrol, a derivative, metabolite or analogue thereof.

15. The use as in claim 1 comprising the use of one or more other active ingredients often used in nutraceutical compositions.

16. A method of promoting the wellness state of a mammal which comprises providing said mammal with an effective amount of resveratrol, a derivative, metabolite or analogue thereof via a nutraceutical composition.
